(19) United States
(12) Patent Application Publication
(10) Pub. No.: US 2012/0029296 A1
(43) Pub. Date: Feb. 2, 2012

(54) EXPANDABLE CANNULA ASSEMBLIES FOR USE IN PERCUTANEOUS SURGICAL PROCEDURES

(75) Inventors: Tanmay Mishra, Mountain View, CA (US); Bryan A. Click, Fremont, CA (US); Bruce Chabansky, Palo Alto, CA (US)

(73) Assignee: KYPHON SARL. A Limited Liability Company, Neuchatel (CH)

(21) Appl. No.: 12/846,544

(22) Filed: Jul. 29, 2010

(51) Int. Cl. A61B 1/32

(52) U.S. Cl. 600/208

(57) ABSTRACT

In one form, an expandable cannula assembly for use in percutaneous surgical procedures includes an elongate body extending along a longitudinal axis between a first end and a second end. The elongate body includes a pair of elongate members that cooperate to define a working channel extending between a proximal end and a distal end and being expandable from a first, unexpanded configuration to a second, expanded configuration. The elongate members are displaceable away from one another along the longitudinal axis from the proximal end to the distal end of the working channel in order to expand the working channel from the first configuration to the second configuration. In one aspect, the elongate members engage with one another to lock the working channel in the second configuration and prevent movement of the elongate members toward one another. However, in other embodiments, different forms and applications are envisioned.

### BACKGROUND

The present application relates to cannula assemblies and methods for using same in performing surgery in a patient, and more particularly, but not exclusively, relates to cannula assemblies that provide expandable working channels.

Traditional surgical procedures for pathologies located within the body involve extensive and lengthy cutting, removing, and or repositioning skin and tissue surrounding the surgical site in order for the surgeon to access the surgical site. This type of approach may cause trauma, damage, and scarring to the tissue, and also presents risks that the tissue will become infected and that a long recovery time will be required after surgery for the tissue to heal. In some cases, these invasive procedures lead to permanent scarring and pain that can be more severe than the pain leading to the surgical intervention.

Minimally invasive surgical techniques are particularly desirable in, for example, spinal and neurosurgical applications because of the need for access to locations deep within the body and the presence of vital intervening tissues. The development of percutaneous minimally invasive spinal procedures has yielded a major improvement in reducing recovery time and post-operative pain because they require minimal, if any, muscle dissection and can be performed under local anesthesia. These benefits of minimally invasive techniques have also found application in surgeries for other locations in the body where it is desirable to minimize tissue disruption and trauma. However, current techniques for minimally invasive surgery can require numerous steps before access is gained to the surgical site which can lead to prolonged retraction of tissues and increased complexity and duration of the surgical procedure, amongst other things. Thus, there remains a need for further improvements in the devices, instruments, assemblies, apparatuses, systems and methods for performing minimally invasive and other surgical techniques.

### SUMMARY

One nonlimiting embodiment of the present application is directed to an expandable cannula assembly for use in percutaneous surgical procedures. The cannula assembly includes an elongate body extending along a longitudinal axis between a first end and a second end. The elongate body includes a pair of elongate members that cooperate to define a working channel extending from a proximal end to a distal end and being expandable from a first, unexpanded configuration to a second, expanded configuration. The elongate members are displaceable away from one another along the longitudinal axis from the proximal end to the distal end of the working channel in order to expand the working channel from the first configuration to the second configuration. In one aspect of this embodiment, the elongate members engage with one another to lock the working channel in the second configuration and prevent movement of the elongate members toward one another.

In another embodiment, an expandable cannula assembly includes an elongate body extending along a longitudinal axis between a first end and a second end. The elongate body includes a first member partially enclosing a first hollow interior and a second member partially enclosing a second hollow interior. The hollow interiors cooperate to define a working channel extending between a proximal end and a distal end. The working channel is expandable along the longitudinal axis from a first, unexpanded configuration where the second member is positioned in the first hollow interior to a second, expanded configuration by laterally displacing the first and second members away from one another from the proximal end to the distal end. In one aspect of this embodiment, at least a portion of the second member extends outwardly from the first hollow interior of the first member.

In yet another embodiment, a cannula assembly includes an elongate body extending along a longitudinal axis between a first end and a second end. The elongate body includes a first elongate member and a second elongate member cooperating to define a working channel extending from a proximal end to a distal end and being expandable from a first, unexpanded configuration to a second, expanded configuration. The first and second elongate members are displaceable from one another along the longitudinal axis from the proximal end to the distal end of the working channel to expand the working channel to the second configuration. In one aspect of this embodiment, the working channel includes a length between the proximal end and the distal end that is greater than a maximum dimension across the working channel in the second configuration. Still, in another aspect of this embodiment, the first elongate member includes a first portion extending about a second portion of the second elongate member from the proximal end to the distal end of the working channel.

In a further embodiment, a method includes providing a first cannula extending between opposite first and second ends and including a pair of elongate members cooperating to define a first working channel extending between a proximal end and a distal end and being expandable from a first, unexpanded configuration to a second, expanded configuration. A first one of the elongate members is positioned in a second one of the elongate members when the first working channel is in the first configuration. The method also includes positioning the first cannula with the first working channel in the first configuration at a location adjacent to a surgical site and displacing the elongate members laterally away from one another from the proximal end to the distal end of the first working channel to expand the first working channel to the second configuration. Displacing the elongate members includes radially expanding the second one of the elongate members to facilitate expulsion of a portion of the first one of the elongate members from the second one of the elongate members. Still, other methods for using expandable cannula assemblies are also provided.

Another embodiment of the present application is a unique system for surgery in a patient. Other embodiments include unique methods, systems, devices, kits, assemblies, equipment, and/or apparatus for use in connection with percutaneous surgical procedures. However, in other embodiments, different forms and applications are envisioned.

Further embodiments, forms, features, aspects, benefits, objects and advantages of the present application shall become apparent from the detailed description and figures provided herewith.

### BRIEF DESCRIPTION OF THE FIGURES

FIG. 1 is a perspective view of one embodiment of an expandable cannula assembly.

FIG. 2 is a section view of the cannula assembly illustrated in FIG. 1 taken along view line 2-2.

FIG. 3 is a perspective view of a separation instrument and the cannula assembly illustrated in FIG. 1 in an expanded configuration.

FIG. 4 is a section view of the cannula assembly illustrated in FIG. 3 taken along view line 4-4.

FIG. 5 is a section view of an alternative embodiment expandable cannula assembly.

FIG. 6 is a side view of a delivery cannula positioned at a location adjacent to a surgical site.

FIG. 7 is a side view of the cannula assembly illustrated in FIG. 1 positioned in the delivery cannula of FIG. 6.

FIG. 8 is a section view taken along view line 8-8 of FIG. 7.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices and described methods, and any such further applications of the principles of the invention as illustrated herein are contemplated as would normally occur to one skilled in the art to which the invention relates.

Instruments, systems, apparatuses, assemblies and methods for performing surgery, including spinal surgeries that include one or more techniques such as laminotomy, laminectomy, foramenotomy, facetectomy, discectomy, interbody fusion, spinal nucleus or disc replacement, and implant insertion including interbody implants and bone engaging fasteners, for example, are provided. The surgery is performed through a working channel or passageway through skin and/or tissue of the patient provided by an expandable cannula assembly. In one form, viewing of the surgical site at the working end of the cannula assembly can be accomplished with naked eye visualization, microscopic viewing devices, loupes, viewing instruments mounted on the cannula, positioned over the cannula, positioned in other portals in the body, and/or through a viewing system such as lateral fluoroscopy. The cannula assembly is movable in situ to increase the size of the working channel to facilitate access to the working space at the distal end of the cannula assembly while minimizing trauma to tissue surrounding the cannula assembly. The cannula assembly can be used with any surgical approach to the spine, including anterior, posterior, posterior mid-line, lateral, postero-lateral, and/or antero-lateral approaches, and in other regions besides the spine.

In one embodiment, an expandable cannula assembly for use in percutaneous surgical procedures includes an elongate body extending along a longitudinal axis between a first end and a second end. The elongate body includes a pair of elongate members that cooperate to define a working channel extending from a proximal end to a distal end and being expandable from a first, unexpanded configuration to a second, expanded configuration. The elongate members are displaceable away from one another along the longitudinal axis from the proximal end to the distal end of the working channel in order to expand the working channel from the first configuration to the second configuration. In one aspect, the elongate members engage with one another to lock the working channel in the second configuration and prevent movement of the elongate members toward one another. Still, it should be appreciated that alternative forms, aspects, configurations, arrangements and methods are contemplated with respect to the subject matter disclosed and described herein.

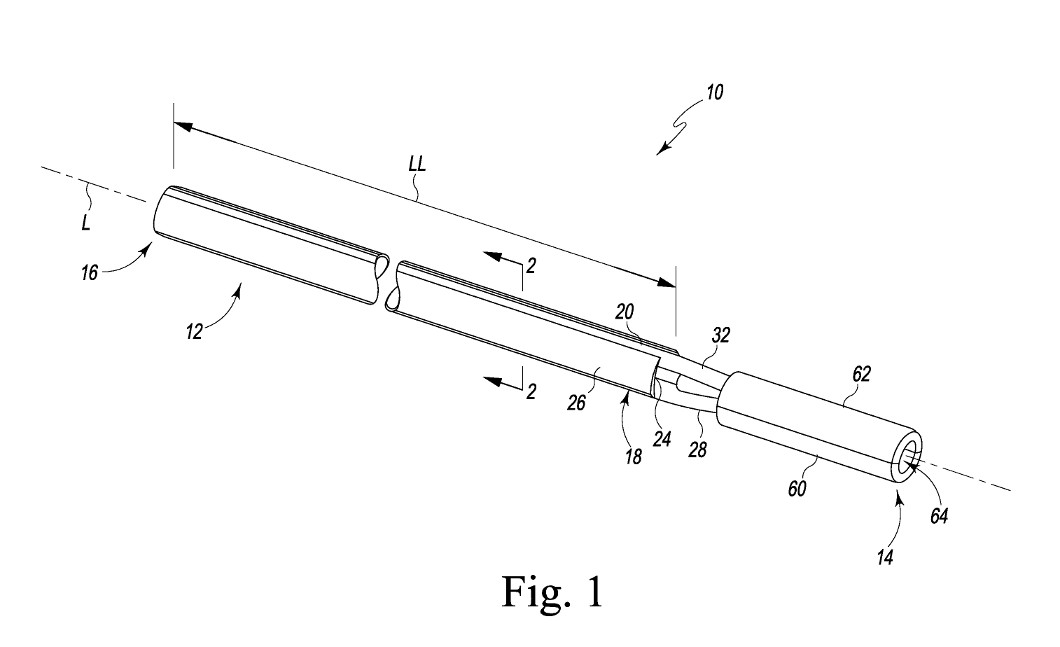

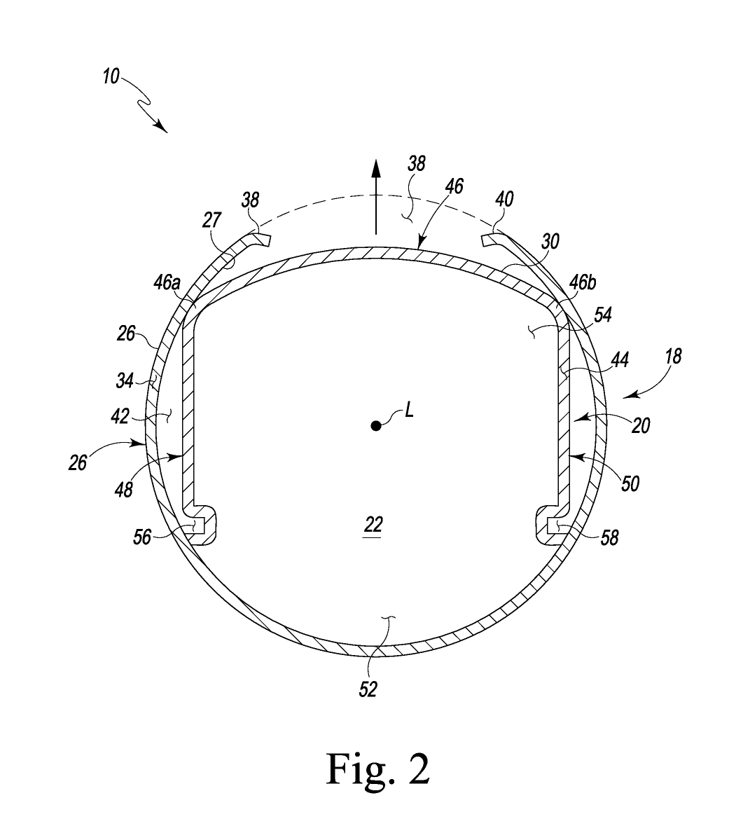

Referring now generally to FIGS. 1-4, further details regarding cannula assembly 10 will be provided. More particularly, as illustrated in the perspective view of FIG. 1, cannula assembly 10 includes an elongate body 12 extending between a proximal end 14 and a distal end 16. Elongate body 12 includes a pair of elongate members 18, 20 that extend between proximal end 14 and distal end 16 and can include beveled ends at distal end 16 to facilitate insertion in an incision, although non-beveled ends are also contemplated. Elongate members 18, 20 cooperate to define and surround an expandable working channel 22 illustrated in FIG. 2 for example. Working channel 22 extends between and opens at distal end 16 of elongate body 12 and to a proximal end 24 positioned distally of proximal end 14 of elongate body 12. Elongate body 12 generally includes a length LL between proximal end 24 and distal end 16 that facilitates positioning proximal end 24 above the skin of a patient when distal end 16 is positioned adjacent to the targeted surgical site.

Elongate member 18 includes a first perimeter length around its exterior surface 26 between distal end 16 of elongate body 12 and proximal end 24 of working channel 22 and a second, smaller perimeter length around its exterior surface 26 between proximal end 24 of working channel 22 and proximal end 14 of elongate body 12 along a stem portion 28. Elongate member 20 also includes a first perimeter length around its exterior surface 30 (shown in FIG. 2) between distal end 16 of elongate body 12 and proximal end 24 of working channel 22 and a second, smaller perimeter length around its exterior surface 30 between proximal end 24 of working channel 22 and proximal end 14 of elongate body 12 along a stem portion 32. In other non-illustrated forms, it is contemplated that elongate body 12 could be provided without stem portions 28, 32 such that the perimeter length around the exterior surfaces 26, 30 is the same from proximal end 24 to distal end 16. In addition, in the form where stem portions 28, 32 are not included, it should be appreciated that the proximal and distal ends of elongate body 12 will correspond to the proximal and distal ends of working channel 22.

As illustrated in FIG. 2, elongate member 18 includes a sidewall 34 having a generally c-shaped configuration in a plane extending orthogonally to longitudinal axis L. However, it should be appreciated that alternative configurations for sidewall 34 are also contemplated. In the illustrated form, sidewall 34 is generally arcuately rounded between a first end portion 38 and an oppositely positioned second end portion 40 and defines an opening 36 positioned between first and second end portions 38, 40 Opening 36 is laterally offset from longitudinal axis L and communicates with a hollow interior 42 that is enclosed and surrounded by sidewall 34 with the exception of opening 36. Moreover, first and second end portions 38, 40 have a linear configuration that extends inwardly into hollow interior 42 relative to the remaining portions of sidewall 34. Stated alternatively, first and second end portions 38, 40 do not continue along the same radial arc as the remainder of sidewall 34.

Elongate member 20 includes a sidewall 44 having a generally u-shaped configuration in a plane extending orthogonally to longitudinal axis L. However, it should be appreciated that alternative configurations for sidewall 44 are also contemplated, including a generally c-shaped configuration for example. In the illustrated form, sidewall 44 includes an arcuately rounded portion 46 extending between and connected to a first linear portion 48 and an oppositely positioned second linear portion 50 by a pair of rounded transition portions 46a, 46b, respectively. Sidewall 44 further defines an opening 52 positioned between first and second linear portions 48, 50 at an end positioned opposite of arcuately rounded portion 46. Opening 52 is laterally offset from longitudinal axis L and communicates with a hollow interior 54 that is enclosed by sidewall 44 with the exception of opening 52. Moreover, first linear portion 48 includes a receptacle 56 that is positioned opposite of arcuately rounded portion 46 and is configured to receive first end portion 38 of sidewall 34 when working channel 22 is enlarged to the expanded configuration. Similarly, second linear portion 50 includes a receptacle 58 that is positioned opposite of arcuately rounded portion 46 and is configured to receive second end portion 40 of sidewall 34 when working channel 22 is enlarged to the expanded configuration. Receptacles 56, 58 generally extend orthogonally to axes upon which the remaining portions of linear portions 48, 50 extend. Further details regarding the engagement of first and second end portions 38, 40 with receptacles 56, 58 will be provided below.

Working channel 22 is illustrated in an unexpanded configuration in FIG. 2. In this configuration, elongate member 20 is housed within hollow interior 42 of elongate member 18 such that sidewall 34 generally surrounds elongate member 20 and opening 36 is positioned outside or laterally of elongate member 20. Moreover, transition portions 46a, 46b and linear portions 48, 50 of sidewall 44 adjacent to receptacles 56, 58 are positioned in engagement with interior surface 27 of sidewall 34 of elongate member 18. In addition, hollow interiors 42, 54 cooperate to define working channel 22 which, in the unexpanded configuration, is surrounded and enclosed by sidewall 44 and a portion of sidewall 34 in a plane extending orthogonally to longitudinal axis L.

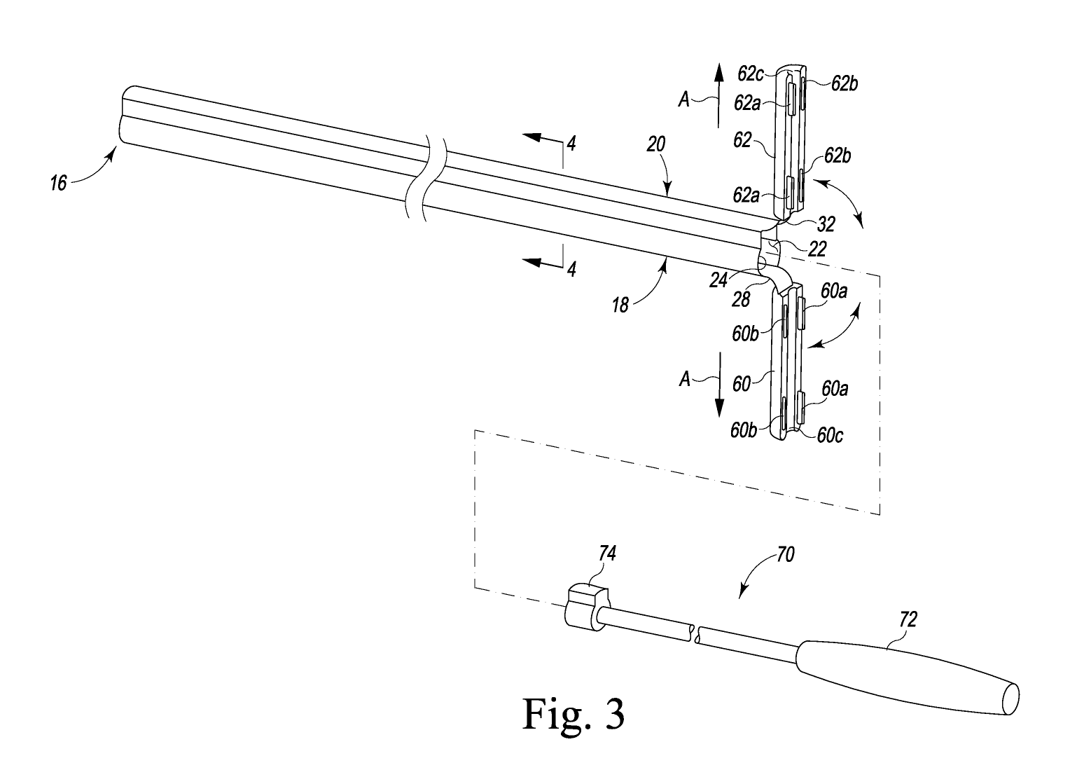

Cannula assembly 10 further includes a first tab member 60 coupled to stem portion 28 of elongate member 18 and a second tab member 62 coupled to stem portion 32. However, in certain embodiments, where stem portions 28, 32 are not included for example, it is contemplated that tab members 60, 62 will not be included by cannula assembly 10. As illustrated in FIG. 3 for example, tab member 60 includes an elongate groove 60c positioned between a pair of projections 60a and a pair of indentations 60b and tab member 62 includes an elongate groove 62c positioned between a pair of projections 62a and a pair of indentations 62b. Projections 60a can be positioned into indentations 62b and projections 62a can be positioned in indentations 60b to provide a snap-fit engagement such that tab members 60, 62 are releasably engageable to one another as illustrated in FIG. 1 for example. However, it should be appreciated that alternative arrangements are contemplated for releasably engaging tab members 60 and 62. Examples of such arrangements include ball-detent mechanisms, fasteners, threaded coupling members, clamping members, snap rings, compression bands, and straps, just to name a few possibilities. When tab members 60, 62 are engaged with one another, elongate grooves 60c and 62c cooperate to define an elongate passage 64 that extends through tab members 60, 62 and between stem portions 28, 32 into communication with proximal end 24 of working channel 22. In addition, the engagement of tab members 60, 62 with one another prevents rotation of elongate members 18, 20 relative to one another and, as illustrated in FIG. 2 for example, maintains alignment of arcuately rounded portion 46 of elongate member 20 with opening 36 of elongate member 18.

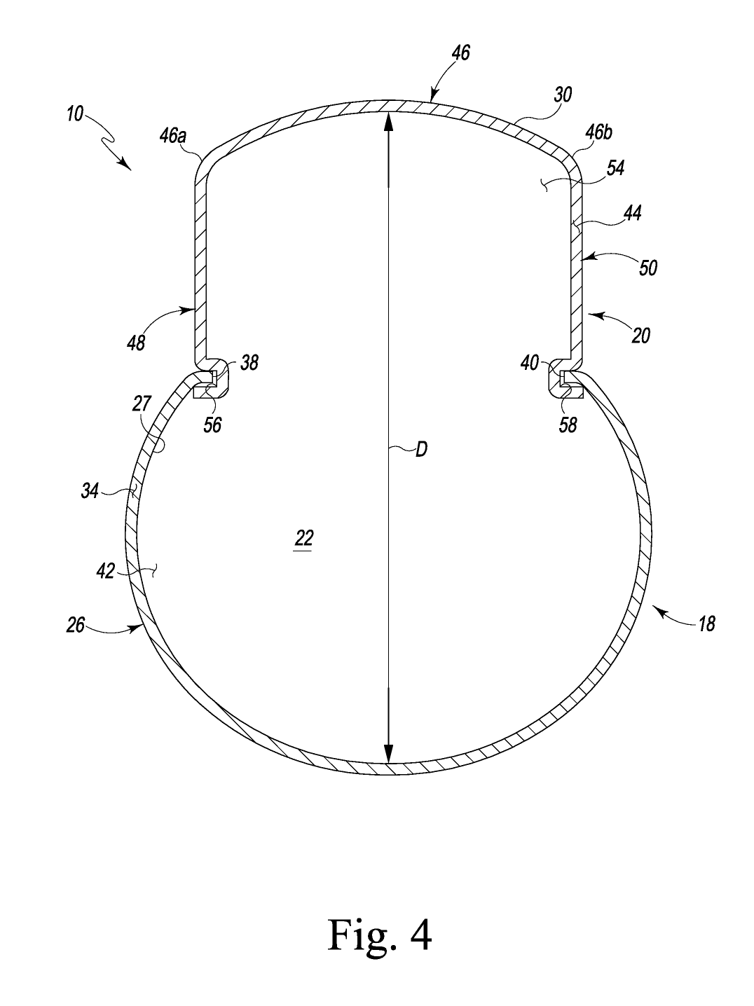

Elongate body 12 of cannula assembly 10 is insertable through an incision in skin and tissue of a patient to provide working channel 22 to a surgical site. It is contemplated that elongate body 12 can initially be inserted through skin and tissue in an insertion configuration for working channel 22 such as the configuration illustrated in FIGS. 1 and 2 for example. Working channel 22 can have a size in the insertion configuration that allows access to the surgical location in the patient's body to perform one or more steps of the surgical procedure. However, it may be desirable during surgery to provide greater access to the surgical site in the patient's body beyond what is provided through working channel 22 in its initial insertion configuration. Similarly, after insertion into the patient, working channel 22 can be enlarged by separating elongate members 18, 20 from one another along longitudinal axis L. Separation of elongate members 18, 20 increases the size of working channel 22 from proximal end 24 to distal end 16. Further details regarding the expansion of working channel 22 will be provided below in connection with FIGS. 3 and 4.

More particularly, in order to expand working channel 22, tab members 60, 62 are first separated from one another. While not illustrated, it should be appreciated that in one form tab members 60, 62 could be separated from one another by distally moving a tapered separation instrument through elongate passage 64 until projections 60a are released from indentations 62b and projections 62a are released from indentations 60b. Once released from engagement with one another, the proximal ends of tab members 60, 62 may be pivoted distally away from the longitudinal axis L to position tab members 60, 62 in the configuration illustrated in FIG. 3. As tab members 60, 62 are pivoted in this manner, stem portions 28, 32, which may generally be more flexible than the remaining portions of elongate members 18, 20 between distal end 16 and proximal end 24 of working channel 22, are bent or deformed adjacent to proximal end 24 of working channel 22. In one form, it is contemplated that a surgeon or other medical professional could separate tab members 60, 62 from one another relative to longitudinal axis L as indicated by directional arrows A in FIG. 3 once they have been pivoted to the illustrated configuration. Alternatively, it is also contemplated that tab members 60, 62 could be separated in this manner as they are pivoted away from the longitudinal axis L.

As tab members 60, 62 are separated from one another as indicated by directional arrows A, elongate members 18, 20 are moved away from one another and transition portions 46a, 46b of elongate member 20 adjacent proximal end 24 of working channel 22 are brought into further engagement with interior surface 27 of elongate member 18. As this occurs, elongate member 18 is radially expanded about longitudinal axis L, thereby increasing the distance across opening 36 and allowing a portion of elongate member 20 to be expelled or displaced from hollow interior 42 of elongate member 18. Upon continued lateral displacement of elongate member 20 from hollow interior 42, first and second end portions 38, 40 of sidewall 34 of elongate member 18 are received in receptacles 56, 58 as illustrated in FIG. 4 for example.

Once working channel 22 is expanded adjacent to proximal end 24, a separation instrument 70 can be inserted into working channel 22 and moved distally therethrough in order to displace elongate member 20 from hollow interior 42 such that working channel 22 has an expanded configuration from proximal end 24 to distal end 16. Moreover, in this configuration, first and second end portions 38, 40 are received in receptacles 56, 58 from proximal end 24 to distal end 16 such that a portion of elongate member 18 extends about or is positioned around elongate member 20 from proximal end 24 to distal end 16. As illustrated in FIG. 4 for example, when working channel 22 is expanded it generally includes a curvilinear configuration and is enclosed and surrounded by elongate members 18, 20 in a plane extending orthogonally to longitudinal axis L. Moreover, in its expanded configuration, the maximum dimension D across working channel 22 is generally less than length LL of elongate body 12, although it should be appreciated that variations in the relationship of dimension D and length LL are also contemplated. Furthermore, it should be appreciated that working channel 22 will generally have a continuous size and shape from proximal end 24 to distal end 16 when it is in the expanded configuration.

While not previously discussed, it should be appreciated that the engagement between first and second end portions 38, 40 and receptacles 56, 58 maintains working channel 22 in the expanded configuration and prevents elongate members 18, 20 from moving toward one another. However, it is contemplated that linear portions 48, 50 of elongate member 20 could be squeezed or moved together in order to disengage first and second end portions 38, 40 from receptacles 56, 58. Once disengaged, elongate members 18, 20 could be forced together to radially expand elongate member 18 and allow elongate member 20 to be repositioned into hollow interior 42. In addition, separation instrument 70 generally includes a handle portion 72 coupled with a head portion 74 which, in the illustrated form, is provided with an external configuration that generally corresponds to the internal configuration of working channel 22 in its expanded configuration. However, it should be appreciated that alternative configurations for head portion 74 are also contemplated provided that such configurations accomplish full expansion of working channel 22. For example, in one form, head portion 74 could simply be provided with a height that corresponds to the maximum dimension D across working channel 22 in its expanded configuration to ensure that working channel 22 is fully expanded as separation instrument 70 is moved distally therethrough. In another form, it is contemplated that head portion 74 could be an implant that is releasably engaged with handle portion 72 and expands working channel 22 as it is inserted therethrough to the surgical site.

In an alternative form for expanding working channel 22, it is contemplated that separation of elongate members 18, 20 could be accomplished by separation instrument 70 alone without separating tab members 60, 62 from one another to initially align first and second end portions 38, 40 with receptacles 56, 58 adjacent to proximal end 24 of working channel 22. For example, it is contemplated that head portion 74 of separation instrument 70 could he provided with a tapered configuration that can be inserted into proximal end 24 of working channel 20 once tab members 60, 62 have been pivoted away from longitudinal axis L. Similarly, in this configuration the tapered head portion 74 could be moved distally to displace elongate members 18, 20 from one another.

In a further aspect, it should be appreciated that tab members 60, 62 can be engaged with one or more operating room support structures when they are pivoted away from longitudinal axis L as illustrated in FIG. 3. More particularly, in one form the support structures can be provided with projections and indentations that can correspondingly engage with projections 60a, 62a and indentations 60b, 62b, although other variations for engaging tab members 60, 62 with the support structures are contemplated. Examples of operating room support structures include, without limitation, support arms, braces and other linkage members which are coupled to an operating table or bed and movable to position assembly 10 relative to a surgical site of the patient.

Elongate members 18, 20 can be provided with sufficient rigidity between distal end 16 and proximal end 24 of working channel 22 to separate and maintain separation of tissue when tissue is retracted by moving elongate members 18, 20 away from one another. For example, elongate members 18, 20 can include a thickness which provides sufficient rigidity to resist bending or bowing under the forces exerted on it by the retracted tissue. Also, the arcuately shaped portions of sidewalls 34, 44 can assist in providing a sufficient section modulus or moment of inertia in the direction of movement of elongate members 18, 20 to resist bending, bowing and/or deflection forces applied during such movement. Furthermore, it is contemplated that elongate members 18, 20 can be made from any biocompatible material, including but not limited to non-reinforced polymers, carbon-reinforced polymer composites, shape-memory alloys, titanium, titanium alloys, cobalt chrome alloys, stainless steel and others as well.

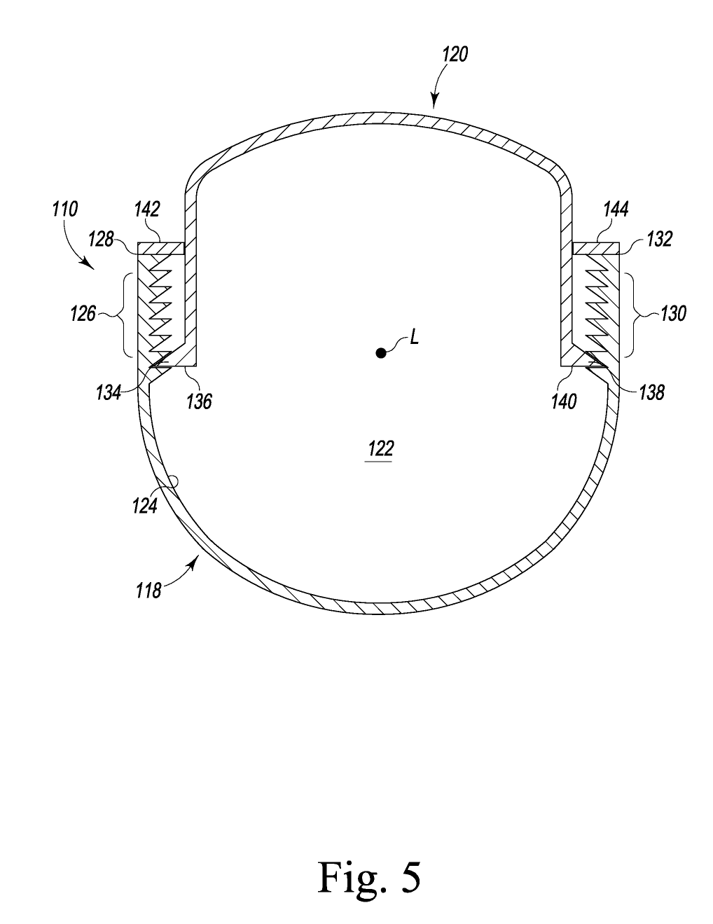

An alternative embodiment cannula assembly 110 is illustrated in section view in FIG. 5. Assembly 110 is generally the same as assembly 10, but includes an alternatively configured arrangement for maintaining the spacing between elongate members 118, 120 when working channel 122 is in the expanded configuration. More particularly, elongate member 118 generally includes a c-shaped configuration in a plane extending orthogonally to longitudinal axis L. Interior surface 124 of elongate member 118 also includes a first plurality of teeth 126 positioned adjacent end portion 128 and a second plurality of teeth 130 positioned adjacent end portion 132. Elongate member 120 also generally includes a c-shaped configuration in a plane extending orthogonally to longitudinal axis L. In addition, elongate member 120 further includes a first laterally extending flange portion 134 positioned adjacent to end portion 136 and a second laterally extending flange portion 138 positioned adjacent to end portion 140. As will be discussed in greater detail below, flange portions 134, 138 are configured to engage with teeth 126, 130, respectively, to control the positioning of elongate members 118, 120 relative to one another.

More particularly, as illustrated in FIG. 5 for example, when flange portions 134, 138 engage with teeth 126, 130, elongate member 120 can be incrementally moved away from elongate member 118 to expand working channel 122 until flange portions 134, 138 contact stop portions 142, 144, which extend beyond teeth 126, 130 and limit further separation of elongate member 120 from elongate member 118. However, unless elongate member 120 is radially compressed, the engagement of flange portions 134, 138 with teeth 126, 130 prevents movement of elongate member 120 toward elongate member 118. More particularly, flange portions 134, 138 of elongate member 120 slip past teeth 126, 130 of elongate member 118 when elongate member 120 is moved away from elongate member 118. However, if elongate member 120 is forced toward elongate member 118, flange portions 134, 138 interlock with teeth 126, 130 and prevent movement of elongate member 120 toward elongate member 118. In other forms, it is contemplated that the positioning of the flange portions and series of teeth could be interchanged between elongate members 118, 120.

While not previously discussed, it should be appreciated that working channel 122 of assembly 110 can be expanded in a manner similar to that described above with respect to working channel 22 of assembly 10. For example, assembly 110 could be inserted with working channel 122 in an unexpanded configuration followed by expansion using a separation instrument to separate elongate members 118, 120 from one another either alone or in combination with initiating separation of elongate members 118, 120 by moving the tab members away from one another as discussed above with respect to assembly 10.

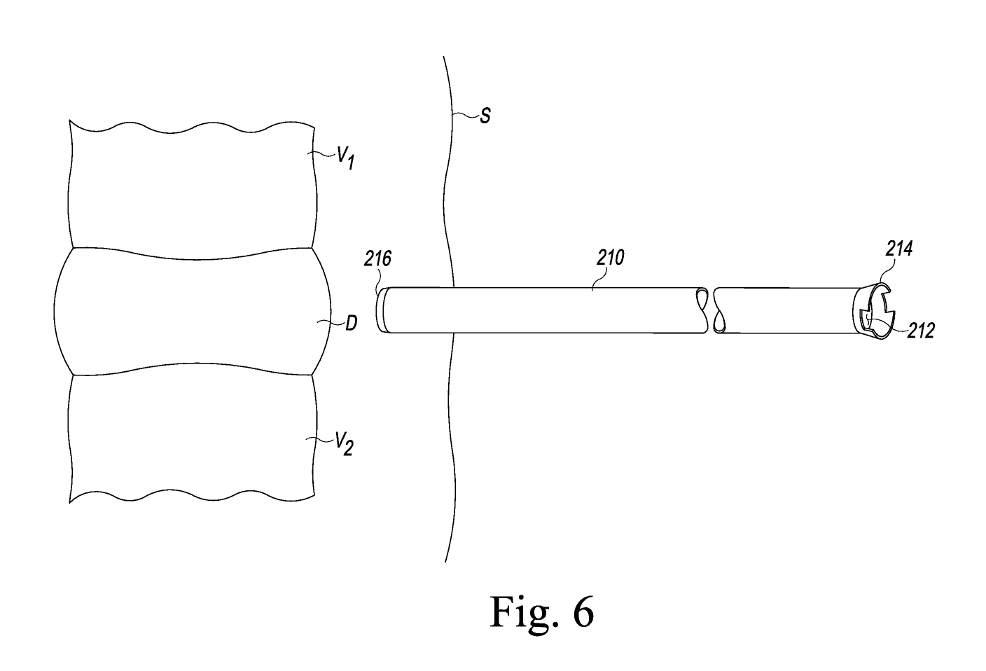

One particular application for cannula assemblies 10, 110 is in spinal surgery. For example, with further reference to FIGS. 6-8, a method of using assembly 10 relative to a disc D positioned between adjacent vertebral bodies V₁ and V₂ will be described. It should be appreciated however that the described method may also be used in connection with assembly 110. In addition, while assembly 10 is described as being used in connection with a surgical procedure performed on or in relation to disc D between adjacent vertebral bodies V₁ and V₂, it should be appreciated that use of assemblies, 10, 110 at other locations along the spinal column and at other anatomical locations besides the spinal column are contemplated.

With reference to FIG. 6 for example, an insertion cannula 210 is inserted through an incision in the skin S and advanced to a location adjacent disc D between adjacent vertebral bodies V₁ and V₂. Cannula 210 generally includes a working channel 212 that extends between and opens at proximal end 214 and distal end 216. It should be appreciated that disc D can be accessed from any of an anterior, posterior, antero-lateral, postero-lateral or lateral approach. In one form, prior to insertion of cannula 210, the skin and tissue can be sequentially dilated via a dilation instrument set (not illustrated) which can include guidewires and/or one or more tissue dilators of increasing size. The tissue dilators are inserted one over another to form a pathway through the skin and tissue to the surgical site in the patient. In such procedures, cannula 210 is positioned over an inserted dilator to form a pathway through the skin and tissue adjacent to disc D, and the guidewires and dilators, if used, are removed from cannula 210 to leave working channel 212 open. However, it should be appreciated that insertion and positioning of cannula 210 without guidewires and dilators is also possible and contemplated in connection with the use of assembly 10. Furthermore, it should also be appreciated that cannula assembly 10 can be positioned directly through the skin and tissue of a patient to a location adjacent a surgical site without the use of cannula 210.

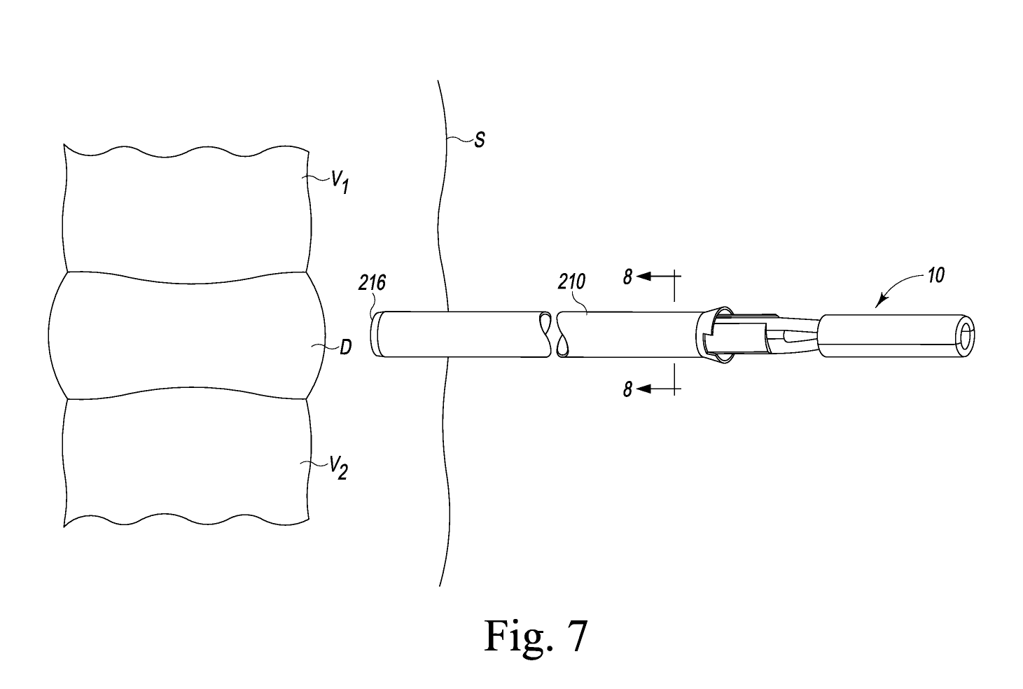

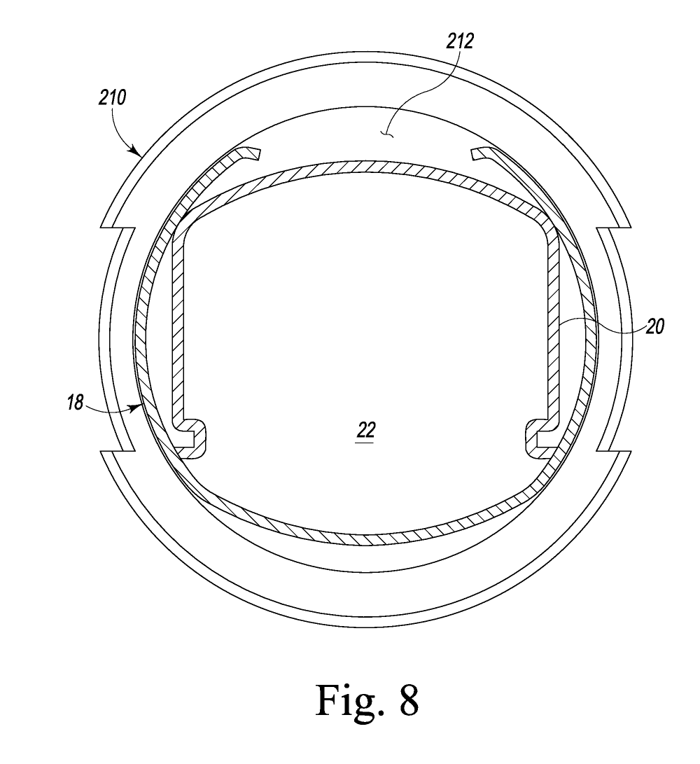

As illustrated in FIG. 7, cannula assembly 10 is positioned in working channel 212 of cannula 210 after it has been properly positioned relative to the surgical site. Once positioned in working channel 212 of cannula 210, cannula assembly 10 still provides access to the surgical site through working channel 22 in its unexpanded configuration. For the entire surgery or for certain procedures during the surgery, it may be desired by the surgeon to increase the size of working channel 22 to facilitate access to the surgical site with differently sized and/or shaped implants or instruments. However, as illustrated in FIG. 8 for example, working channel 212 is sized relative to elongate member 18 such that it prevents radial expansion of elongate member 18, which in turn prevents separation of elongate members 18, 20 from one another to expand working channel 22. Similarly, cannula 210 is removed from the incision over cannula assembly 10 in order to allow elongate members 18, 20 to he separated along longitudinal axis L as discussed above to increase the size of working channel 22 and provide a working space larger than that provided by working channel 212. In one form, cannula assembly 10 can be positioned relative to disc D such that elongate members 18, 20 can be primarily or predominantly separable in the direction of the spinal column axis since the muscle tissue adjacent the spine has a fiber orientation that extends generally in the direction of the spinal column axis. The separation of elongate members 18, 20 can also separate the muscle tissue along the fibers, thus the amount of separation and the resultant tearing and trauma to the muscle tissue can be minimized. It is also contemplated in other techniques employing cannula assembly 10 that working channel 22 can be enlarged primarily in a direction other than along the spinal column axis or in areas other than spine.

Upon completion of the surgical procedure, cannula assembly 10 can be disengaged from any operating room support structures, if utilized, and then removed from its location adjacent to the surgical site with working channel 22 in its expanded configuration. However, it is also contemplated that working channel 22 could be reduced to its unexpanded configuration as discussed above before cannula assembly 10 is removed from its location adjacent to the surgical site.

Alternative configurations and uses of the cannula assemblies described herein are also contemplated. For example, in one form, one or more additional cannula assemblies could be positioned at one or more other vertebral levels of the spinal column in order to perform a surgical procedure across multiple levels of the spinal column. In addition, the cannula assemblies and methods described herein may also be used in surgical procedures involving animals, or in demonstrations for training, education, marketing, sales and/or advertising purposes. In addition, the cannula assemblies and methods described herein may also be used on or in connection with a non-living subject such as a cadaver, training aid or model, or in connection with testing of surgical systems, surgical procedures, orthopedic devices and/or apparatus.

Any theory, mechanism of operation, proof, or finding stated herein is meant to further enhance understanding of the present application and is not intended to make the present application in any way dependent upon such theory, mechanism of operation, proof, or finding. It should be understood that while the use of the word preferable, preferably or preferred in the description above indicates that the feature so described may be more desirable, it nonetheless may not be necessary and embodiments lacking the same may be contemplated as within the scope of the application, that scope being defined by the claims that follow. In reading the claims it is intended that when words such as “a,” “an,” “at least one,” “at least a portion” are used there is no intention to limit the claim to only one item unless specifically stated to the contrary in the claim. Further, when the language “at least a portion” and/or “a portion” is used the item may include a portion and/or the entire item unless specifically stated to the contrary.

While the application has been illustrated and described in detail in the drawings and foregoing description, the same is to be considered as illustrative and not restrictive in character, it being understood that only the selected embodiments have been shown and described and that all changes, modifications and equivalents that come within the spirit of the application as defined herein or by any of the following claims are desired to be protected.

What is claimed is:

1. An expandable cannula assembly, comprising an elongate body extending along a longitudinal axis between a first end and a second end, said elongate body including a first member partially enclosing a first hollow interior and a second member partially enclosing a second hollow interior, wherein said first and second hollow interiors cooperate to define a working channel extending between a proximal end and a distal end and being expandable along said longitudinal axis from a first, unexpanded configuration where said second member is positioned in said first hollow interior to a second, expanded configuration by laterally displacing said first and second members away from one another from said proximal end to said distal end.

2. The assembly of claim 1, wherein said working channel is enclosed by said first and second members in a plane extending transversely to said longitudinal axis.

3. The assembly of claim 1, wherein said first and second members interlock with one another to maintain said working channel in said second, expanded configuration.

4. The assembly of claim 1, wherein said first member is radially expandable about said longitudinal axis to facilitate lateral displacement of said second member.

5. The assembly of claim 1, wherein said first member includes a substantially c-shaped sidewall in a plane extending transversely to said longitudinal axis, said substantially c-shaped sidewall defining a lateral opening communicating with said first hollow interior.

6. The assembly of claim 5, wherein said lateral opening is positioned laterally of said second member when said working channel is in said first, unexpanded configuration.

7. The assembly of claim 6, wherein a portion of said second member extends through and is positioned laterally of said lateral opening when said working channel is in said second, expanded configuration.

8. The assembly of claim 5, wherein said second member includes a substantially u-shaped sidewall in a plane extending transversely to said longitudinal axis, said substantially u-shaped sidewall including an arcuate portion extending between a pair of linear portions.

9. The assembly of claim 8, wherein said substantially c-shaped sidewall includes an arcuate portion extending between a first end portion and a second end portion and said linear portions of said substantially u-shaped sidewall of said second member include receptacles configured to receive said first and second end portions of said substantially c-shaped sidewall when said working channel is in said second, expanded configuration.

10. The assembly of claim 1, wherein said proximal end of said elongate body includes a first tab coupled with said first member and a second tab coupled with said second member, said first and second tabs being releasably engageable with one another along said longitudinal axis.

11. The assembly of claim 10, wherein said first and second members are flexible adjacent to said first and second tabs to facilitate pivotal movement of said tabs away from said longitudinal axis in a distal direction.

12. The assembly of claim 10, wherein engagement of said first and second tabs to one another prevents rotation of said first and second members relative to each other.

13. A cannula assembly, comprising an elongate body extending along a longitudinal axis between a first end and a second end, said elongate body including a first elongate member and a second elongate member cooperating to define a working channel extending from a proximal end to a distal end and being expandable from a first, unexpanded configuration to a second, expanded configuration, said first and second elongate members being displaceable from one another along said longitudinal axis from said proximal end to said distal end of said working channel to expand said working channel to said second configuration, wherein said working channel includes a length between said proximal end and said distal end that is greater than a maximum dimension across said working channel in said second configuration, and wherein said first elongate member includes a first portion extending about a second portion of said second elongate member from said proximal end to said distal end of said working channel.

14. The assembly of claim 13, wherein said first portion of said first elongate member engages with said second portion of said second elongate member to maintain said working channel in said second configuration.

15. The assembly of claim 14, wherein one of said first and second portions includes a series of teeth and the other of said first and second portions includes a flange movable along and engageable with said series of teeth.

16. The assembly of claim 14, wherein said second portion of said second elongate member includes a pair of opposing receptacles in which oppositely positioned flanges of said first portion of said first elongate member are positioned when said working channel is in said second configuration.

17. The assembly of claim 14, wherein in said second configuration said working channel is enclosed by said first and second elongate members in a plane extending transversely to said longitudinal axis and includes a curvilinear configuration in said plane.

18. A method, comprising:
providing a first cannula extending between opposite first and second ends and including a pair of elongate members cooperating to define a first working channel extending between a proximal end and a distal end and being expandable from a first, unexpanded configuration to a second, expanded configuration, wherein a first one of said elongate members is positioned in a second one of said elongate members when said first working channel is in said first configuration;
positioning said first cannula with said first working channel in said first configuration at a location adjacent to a surgical site;
displacing said elongate members laterally away from one another from said proximal end to said distal end of said first working channel to expand said first working channel to said second configuration, said displacing including radially expanding said second one of said elongate members to facilitate expulsion of a portion of said first one of said elongate members from said second one of said elongate members.

19. The method of claim 18, which further includes locking said elongate members relative to one another to maintain said first working channel in said second configuration.

20. The method of claim 18, further comprising:
providing a second cannula including a second working channel;
positioning said second cannula at said location, wherein positioning said first cannula at said location includes inserting said first cannula through said second working channel of said second cannula; and
removing said second cannula from said location before displacing said elongate members laterally away from one another.
